(19) United States
(12) Patent Application Publication
(10) Pub. No.: US 2008/0320610 A1
(43) Pub. Date: Dec. 25, 2008

(54) Transgenic fish with increased unsaturated fatty acid content

(76) Inventors: Goro Yoshizaki, Tokyo (JP); Toshiro Takeuchi, Chigasaki-shi (JP); Shuichi Sato, Tokyo (JP); Viswanath Kiron, Bodo (NO); Alimuddin, Bogor (ID)

Correspondence Address: FROMMER LAWRENCE & HAUG, 745 FIFTH AVENUE- 10TH FL., NEW YORK, NY, 10151, US

(21) Appl. No.: 11/541,375

(22) Filed: Sep. 29, 2006

Related U.S. Application Data
(63) Continuation-in-part of application No. PCT/JP05/05688, filed on Mar. 28, 2005.

(30) Foreign Application Priority Data
Mar. 31, 2004 (JP) 2004-108330

(51) Int. Cl. A01K 67/027

(52) U.S. Cl. 800/20

(57) ABSTRACT

The present invention relates to fish with increased unsaturated fatty acid content by transfer of a fatty acid desaturase gene thereinto, and a production method of these fish with increased unsaturated fatty acid content. The fatty acid desaturase gene is one or more members selected from among Δ4 fatty acid desaturase gene, Δ5 fatty acid desaturase gene and Δ6 fatty acid desaturase gene, and it is preferable that these genes originate in a freshwater fish. It is preferable that the expression of a fatty acid desaturase gene originating in a freshwater fish is promoted by a β-actin promoter originating in medaka. By modifying the fatty acid metabolic pathway of fish, problems to be solved by the present invention may be to provide fish having a high vitality and a tolerance to handling and low temperature, which will enable the use of a vegetable fat or animal fat as a formula feed. Such vegetable fat is less expensive and can be easily controlled in qualities, thereby largely contributing to the reduction of cost and labor in fish nursery sites.

### INCORPORATION BY REFERENCE

This application is a continuation-in-part application of international patent application Serial No. PCT/JP2005/005688 filed Mar. 28, 2005, and published as international publication no. WO 2005/094570 on Oct. 13, 2005, which claims priority to Japanese patent application Serial No. JP 2004-108330 filed Mar. 31, 2004.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates to fish with increased unsaturated fatty acid content by transfer of a fatty acid desaturase gene thereinto, and a production method of these fish with increased unsaturated fatty acid content.

### BACKGROUND OF THE INVENTION

The beneficial effect of n-3 highly unsaturated fatty acids (HUFA), especially eicosapentaenoic acid (EPA, 20:5n-3) and docosahexaenoic acid (DHA, 22:6n-3), on human health and development have been extensively studied (for instance, see Non-patent literatures 1, 2, 3, 4, 5). In human, these fatty acids are taken with mainly marine fish or its extract, fish oil. In recent years, however, the availability of raw materials for EPA and DHA production has become increasingly scarce due to a decline in the number of fish captured in the wild (for instance, see Non-patent literature 6). Moreover, these supplies are expected to become critical between 2005 and 2010 (for instance, see Non-patent literature 7). Increase in fish production by means of mariculture production could potentially offset the effects of the decline of EPA and DHA supplies from fish captured in the wild. However, the synthesis of desaturated fatty acid in fish depends on their type and the activity of their fatty acid metabolic enzymes. Freshwater fish have enzymes that metabolize linoleic acid to EPA or DHA. In contrast marine fish do not have enzymes that metabolize linoleic acid to EPA or DHA, which is needed for their growth and development (for instance, see Non-patent literatures 8, 9, 10, 11). Instead, marine fish acquire EPA and DHA through their feed. As an example, EPA and DHA in bluefish originate in plankton and, in bluefish cultivation, a formula feed high in EPA and DHA content is needed (for instance, see Non-patent literatures 9, 11). Therefore, an alternative strategy, perhaps utilizing advancements in biotechnology, could be employed to provide a means for producing EPA and DHA in equal or higher levels than those contained in marine fish. Furthermore, this strategy may be less costly compared to providing EPA and DHA to fish in nurseries through their feed.

Many studies on these alternative strategies have demonstrated that biosynthesis of EPA in vertebrates, including fish, occurs by the conversion of dietary α-linolenic acid (18:3n-3) by pathways combining the sequential action of Δ6 and Δ5 desaturation with chain elongation reactions (for instance, see Non-patent literatures 12, 13, 14). Furthermore, it has been described that DHA is synthesized from EPA by the sequential chain elongation to 22:5n-3 and thence to 24:5n-3, followed by a Δ6-desaturation to 24:6n-3, which is finally retroconverted by peroxisomes via β-oxidation (for instance, see Non-patent literatures 12, 15, 16, 17). A synthesis pathway of a single step for DHA that is directly converted from 22:5n-3 by a Δ4 desaturation in marine organism is also known (for instance, see Non-patent literature 18).

Recent progresses in transgenic technology have made it possible to produce animals possessing foreign proteins that are beneficial for human health (for instance, see Non-patent literature 19). This technology has also been applied to aquaculture (for instance, see Non-patent literatures 20, 21, 22). It was demonstrated that transgenic fish can be produced by microinjection into the germinal vesicle of oocyte (for instance, see Non-patent literature 23) or into the cytoplasm of a one-cell stage embryo (for instance, see Non-patent literature 24), electroporation using early embryos (for instance, see Non-patent literature 25) or sperm (for instance, see Non-patent literature 26), retroviral infection (for instance, see Non-patent literatures 27, 28), or a particle gun bombardment (for instance, see Non-patent literature 29). All these methods have successfully facilitated the delivery of a transgene in certain species of fish; however, a suitable method should be selected for each species (for instance, see Non-patent literature 22). Applications of transgenic technology in fish have been established in order to improve growth rate, disease resistance, and adaptation to extreme environmental conditions (for instance, see Non-patent literatures 21, 30).

However, there have been very few studies conducted for targeted modification of fish metabolism pathway using transgenic techniques. In one such study, the potential of gene transfer to compensate for the absence of vitamin C biosynthesis in fish is reported (for instance, see Non-patent literature 31). This method involved the introduction of the L-guluno-γ-lactone oxidase (rGLO) gene, which acted as a catalyst for the terminal reaction for vitamin C. However, no vitamin C production was observed. The improvement of carbohydrate metabolism in rainbow trout by co-injection of a construct containing human glucose transport proteins (hGluT) and rat hexokinase II (rHKII) cDNAs has also been attempted. However, no direct evidence for functionality of rHKII and hGluT1 was observed (for instance, see Non-patent literature 32).

In this experiment described here, a Δ6 desaturase-like gene isolated from yamame salmon (Oncorhynchus masou) as transferred into zebrafish in order to modify fatty acid biosynthesis pathways. The Δ6 desaturase gene was initially targeted due to the fact that this gene is generally considered to be the rate-limiting step in HUFA biosynthesis (for instance, see Non-patent literatures 33, 34).

Non-patent literature 1: Am. J. Clin. Nutr., 54:438-463, 1991

Non-patent literature 2: Nutrition, 16:143-145, 2000

Non-patent literature 3: Nutrition, 16:680-684, 2000

Non-patent literature 4: Prog. Lipid Res, 40:1-94, 2001

Non-patent literature 5: Nutrition, 18:178-188, 2002

Non-patent literature 6: Proc. Nutr. Soc., 58:377-383, 1999

Non-patent literature 7: Aquaculture (In Press), 2002

Non-patent literature 8: J. World Aquacult. Soc., 24:152-161, 1993

Non-patent literature 9: J. Appl. Ichthyol., 11:183-198, 1995

Non-patent literature 10: Aquaculture, 179:217-229, 1999

Non-patent literature 11: Aquaculture, 200:203-222, 2001

Non-patent literature 12: Biochim. Biophys. Acta, 1299:235-244, 1996

Non-patent literature 13: Prog. Lipid Res., 37:73-117, 1998

Non-patent literature 14: Biochim. Biophys. Acta, 1486:219-231, 2000

Non-patent literature 15: Biochem. Physiol, 116B:263-267, 1997

Non-patent literature 16: FEBS Letters, 431:1-6, 1998

Non-patent literature 17: Biochim. Biophys. Acta, 1486:219-231,

Non-patent literature 18: J. Biol. Chem., 276:31561-31566

Non-patent literature 19: Transgenic Res., 9:305-320, 2000

Non-patent literature 20: Biochemistry and molecular biology of fishes, vol. 2., pp: 207-240, 1993

Non-patent literature 21: Aquaculture, 197:191-204, 2001

Non-patent literature 22: Suisanzoshoku, 49:137-142, 2001

Non-patent literature 23: Cell Differen., 19:237-244, 1986

Non-patent literature 24: Aquaculture, 51:143-150, 1986

Non-patent literature 25: Mol. Mar. Biol. Biotechnol., 1:301-308,

Non-patent literature 26: Aquaculture, 173:297-307, 1999

Non-patent literature 27: Science, 265:666-668, 1994a

Non-patent literature 28: Proc. Natl. Acad. Sci. USA., 93:7777-7782, 1996

Non-patent literature 29: FEBS Letters, 287:118-120, 1991.

Non-patent literature 30: Aquaculture, 204:255-269, 2002

Non-patent literature 31: Biochem. Biophys. Res. Commun., 223:650-653, 1996

Non-patent literature 32: Aquaculture, 173:319-332, 1999a

Non-patent literature 33: Prog. Lipid Res., 20:13-22, 1981.

Non-patent literature 34: J. Biol. Chem., 274:471-477, 1999

There are a few examples of technologies related to gene expression of fatty acid desaturase. For instance, animals were transformed to contain fatty acid desaturase, which led to the expression of fatty acid desaturase and the resulting increase in desaturated fatty acid content (as an example, see Patent document 1). Also, a gene encoding Δ9 desaturase from Anacystis that desaturates Δ9 position of fatty acid bound to lipid was transferred into higher plant cells (for instance, see Patent document 2). In another example, Δ5-fatty acid desaturase and its functional portion was isolated from animals (for instance, see Patent document 3). Finally, a method for the production of stearidonic acid in seed was developed, which includes a process to grow plants incorporated with the first DNA construct in the plant genome having functional promoter in plant seed cells, DNA sequence encoding Δ6-desaturase and functional transcription termination region in plant seed with the transcriptional orientation from 5′ to 3′, and a process to survive the plant under the condition in which Δ6-fatty acid desaturase is expressed (for instance, see Patent document 4).

Patent document 1: Unexamined Patent Publication No. 2003-245070

Patent document 2: Unexamined Patent Publication No. 11-332408

Patent document 3: Unexamined Patent Publication No. 14-508932

Patent document 4: Unexamined Patent Publication No. 14-517255

Citation or identification of any document in this application is not an admission that such document is available as prior art to the present invention.

### SUMMARY OF THE INVENTION

Conventionally, fish oil such as EPA and DHA has been needed in the formula feed for marine fish. By modifying the fatty acid metabolic pathway of fish, problems to be solved by the present invention may be to provide fish having a high vitality and a tolerance to handling and low temperature, and its method of production. This will enable the use of a vegetable fat or animal fat as a formula feed which are less expensive and can be easily controlled in qualities, thereby largely contributing to the reduction of cost and labor in fish nursery sites. Problems to be solved by the present invention is also to provide fish containing increased DHA content as a functional food and its method of production.

As an initial step in the present invention, selection of an appropriate construct that allows high Δ6 desaturase-like gene expression was conducted, as efficient transcription of foreign genes in host individuals is one of the important factors involved in introducing new phenotype encoded by the foreign gene (Hacket, 1993; Houdebine, 2000). The teleostean zebrafish, Danio rerio (Westerfield, 1995; Gong, 1999) was employed as a model fish, because it offers several advantages such as a relatively short generation time of 2-3 months, a large number of eggs, and ease of maintaining. We found that when Δ6-desaturase-like cDNA from masou salmon (Oncorhynchus masou) was transferred to zebrafish under regulation of the medaka (Oryzias latipes) β-actin promoter, it was expressed mainly in muscle and the content of EPA and DHA was increased, thereby confirming that Δ6-desaturase-like gene was actually the gene having Δ6 desaturase activity. Thus, the invention was completed.

Namely, the present invention may relate to (1) transgenic fish with increased unsaturated fatty acid content, wherein a fatty acid desaturase gene may be introduced into the transgenic fish and the fatty acid desaturase gene may be expressed therein, (2) the transgenic fish according to (1) wherein the fatty acid desaturase gene may be connected downstream of β-actin promoter derived from medaka, (3) the transgenic fish according to (1) or (2) wherein the fatty acid desaturase gene may be connected upstream of bovine growth hormone polyadenylation sequence, (4) the transgenic fish according to any one of (1) to (3) wherein the fatty acid desaturase gene may consist of any one of the following (A) to (F) sequences;

(A) A nucleic acid sequence (base sequence) encoding a protein that may consist of the amino acid sequence shown by SEQ ID NO: 2;

(B) A nucleic acid sequence that may consist of amino acid sequence with deletion, substitution or addition of one or more amino acids in the amino acid sequence shown by SEQ ID NO: 2 and that may encode a protein having fatty acid desaturase activity;

(C) A nucleic acid sequence that may encode a protein consisting of amino acid sequence having no less than 60% of homology with the amino acid sequence shown by SEQ ID NO: 2 and having a fatty acid desaturase activity;

(D) The nucleic acid sequence shown by SEQ ID NO: 1;

(E) A nucleic acid sequence that may consist of nucleic acid sequence with deletion, substitution or addition of one or more bases in the nucleic acid sequence shown by SEQ ID NO: 1 and that may encode a protein having fatty acid desaturase activity;

(F) A nucleic acid sequence that may hybridize to the nucleic acid sequence shown by SEQ ID NO: 1 under the stringent conditions and that may encode a protein having a fatty acid desaturase activity, (5) Transgenic fish according to any one of (1) to (4) wherein the unsaturated fatty acid may be eicosapentaenoic acid (EPA) or docosahexaenoic acid (DHA), (6) transgenic fish according to any one of (1) to (5) wherein the fish may be cultured.

The invention may enable the utility of a vegetable fat or animal fat, which is less expensive and can be easily controlled in qualities, thereby largely contributing to the reduction of cost and labor in fish nursery sites. Moreover, the invention may enable the production of fish fry having a high vitality and a tolerance to handling and low temperature without resorting to fish oil containing EPA and DHA as a formula feed for fish cultivation. In addition, since fish produced by the invention contain a larger content of DHA than usual, fish production having added value as health food can be realized using closed recirculating system for fish culture. Furthermore, not only for direct use in aquaculture, it can be used as a very useful study tool for elucidating the fatty acid metabolic mechanism in fish at a molecular level, thereby largely contributing to the field of art.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. 112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawing, in which:

FIG. 1 is a figure showing a summary of desaturase gene construct inserted to an expression vector used for production of fish with increased unsaturated fatty acid content of the invention.

FIG. 2 is a figure showing a result of transient expression of desaturase gene by RT-PCR.

FIG. 3 is a figure showing a result of desaturase gene expression in fin, liver and muscle in cDNA of F1 generation by RT-PCR.

FIG. 4 is a figure showing representative results of the germline transmitter screening by PCR analysis with DNA template extracted from pooled-larvae. DNA was extracted from about twenty 2-day-old larvae obtained by crossing the DNA-positive F0 with non-transgenic individuals. Lanes 1 through 6; PCR product amplified using genomic DNA samples from transgenic F0 individuals, Lane C; PCR product amplified using non-transgenic fish, Lane N; PCR product amplified without DNA template, Lane P; amplified pActD6 plasmid DNA (0.6 pg).

FIG. 5 is a figure showing a result of detection of transgene expression in different F1 generation by RT-PCR. Total RNA was extracted from caudal fin tissue. Numbers at the end of construction name indicate individual transgenic line.

FIG. 6 is a figure showing content of desaturated product in fish with increased unsaturated fatty acid content of the invention.

### DETAILED DESCRIPTION

Transgenic fish of the present invention is not limited as long as they are fish with increased unsaturated fatty acid content in which a fatty acid desaturase gene is expressed due to the transfer of the fatty acid desaturase gene. As the fatty acid desaturase gene, their origin is not specially limited and includes animals or vegetables. However, fatty acid desaturase genes such as Δ4 fatty acid desaturase gene, Δ5 fatty acid desaturase gene and Δ6 fatty acid desaturase gene are preferred examples, and they can be used alone or in combination. These Δ4 fatty acid desaturase gene, Δ5 fatty acid desaturase gene and Δ6 fatty acid desaturase gene forms unsaturated bond on 4th, 5th and 6th carbon from the carbon on the carboxyl terminus (delta end) of fatty acid, respectively. Regarding these fatty acid desaturase genes, the ones originating in freshwater fish or plankton are preferred because they do not affect normal development and function in fish. As examples, Δ4 fatty acid desaturase gene from microalga (Tonon, T., Harvey, D., Larson, T. R., and Graham, I. A. Identification of a very long chain polyunsaturated fatty acid Δ4-desaturase from the microalga Pavlova lutheri. FEBS Lett. 553, 440-444 (2003).), Δ5 fatty acid desaturase gene from Atlantic salmon (GenBank: AF478472) or Δ6 fatty acid desaturase gene from yamame salmon (GenBank: AB074149, AB070444).

As preferred examples of Δ6 fatty acid desaturase gene, it is not limited as long as it is a gene consisted of any one of following sequences: (A) A nucleic acid sequence encoding the amino acid sequence shown by SEQ ID NO: 2 (amino acid sequence of Δ6 fatty acid desaturase from yamame salmon); (B) A nucleic acid sequence that encodes an amino acid sequence with deletion, substitution or addition of one or more amino acids in the amino acid sequence shown by SEQ ID NO: 2 and having a fatty acid desaturase activity; (C) A nucleic acid sequence that encodes an amino acid sequence having no less than 60% of homology with the amino acid sequence shown by SEQ ID NO: 2 and having a fatty acid desaturase activity; (D) The nucleic acid sequence shown by SEQ ID NO: 1 (nucleic acid sequence of Δ6 fatty acid desaturase from yamame salmon); (E) A nucleic acid sequence that consists of a nucleic acid sequence with deletion, substitution or addition of one or more bases in the nucleic acid sequence shown by SEQ ID NO: 1 and that encodes a protein having fatty acid desaturase activity; or (F) A nucleic acid sequence that hybridizes to the sequence shown by SEQ ID NO: 1 under stringent conditions and that encodes a protein having a fatty acid desaturase activity. Here, a “nucleic acid sequence encoding a protein having a fatty acid desaturase activity” refers to nucleic acid sequences encoding proteins involved in any action to form unsaturated fatty acid in the body of fish; however, its specific mechanism of action is not particularly limited.

The “amino acid sequence with deletion, substitution or addition of one or more amino acids” refers to amino acid sequences with any amino acids of, for example 1-20, preferably 1-15, more preferably 1-10, more preferably 1-5 are deleted, substituted or added. In addition, a “nucleic acid sequence with deletion, substitution or addition of one or more base” refers to nucleic acid sequences with any bases of, for example 1-20, preferably 1-15, more preferably 1-10, more preferably 1-5 are deleted, substituted or added.

For example, DNA (mutant DNA) consisted of these nucleic acid sequences with one or more bases are deleted, substituted or added may be prepared according to any method known in the art such as. but not limited to, chemical synthesis, genetic engineering method and mutagenesis. Specifically, a mutant DNA can be obtained by the methods in which DNA consisted of nucleic acid sequence shown by SEQ ID NO: 1 is contacted with drug having mutagenicity, is radiated with ultraviolet light, or is introduced a mutation using genetic engineering method, and so on. A site-specific mutagenesis, one of the genetic engineering methods, is useful because specific mutation can be introduced to a specific location, and it can be carried out according to a method described in “Molecular Cloning: A laboratory Manual, 2nd Ed., Cold Spring Harbor Laboratory, Cold Spring Harbor, N.Y., 1989” (hereinafter referred to as “Molecular Cloning 2nd Ed.”) and Current Protocols in Molecular Biology, Supplement 1-38, John Wiley & Sons (1987-1997). By expressing this mutant DNA using a suitable expression line, a protein consisted of an amino acid sequence with deletion, substitution or addition of one or more amino acids can be obtained.

The “amino acid sequence having no less than 60% of homology with the amino acid sequence shown by SEQ ID NO: 2” is not limited as long as the homology of the sequence with amino acid sequence shown by SEQ ID NO: 2 is not less than 60%, and for example the homology is more than 60%, preferably more than 70%, more preferably more than 80%, furthermore preferably more than 90%, specially preferably more than 95%, most preferably more than 98%.

The “nucleic acid sequence that hybridizes under stringent conditions” refers to nucleic acid sequences obtained by using nucleic acids such as DNA or RNA as a probe, and by colony-hybridization, plaque-hybridization or Southern blot hybridization. Specifically, DNA that can be identified by hybridization using a filter immobilized with DNA or fragment of the DNA originating in colony or plaque in the presence of 0.7-1.0 M NaCl at 65° C., and by washing of a filter using a 0.1-2×SSC solution (composition of 1×SSC solution is 150 mM sodium chloride, 15 mM sodium citrate) under condition of 65° C. Hybridization can be conducted according to the methods described “Molecular Cloning 2nd. Ed.”

For example, for DNA that can hybridize under stringent conditions, DNA having a certain level of homology with the nucleic acid sequence of DNA which is used as a probe. DNA having, for example, more than 60%, preferably more than 70%, more preferably more than 80%, furthermore preferably more than 90%, specially preferably more than 90%, most preferably more than 98% of homology is suitably specified.

Methods for obtaining and preparing a gene of the invention are not specially limited. A gene of the interest can be isolated by preparing an appropriate probe or primer according to the amino acid sequence or nucleic acid sequence information shown by SEQ ID NO: 1 or SEQ ID NO: 2 disclosed in the present specification and by screening a cDNA library which is expected to contain the gene, or a gene of the interest can be prepared by chemical synthesis according to a standard method.

Specifically, a gene of the invention can be obtained by selecting a gene of the interest from the cDNA library which has been prepared according to an ordinary method by using an appropriate probe specific to the genes of the invention from yamame salmon from which a gene of the invention has been isolated. The origin of the cDNA can be various cells and tissues originating in the vegetables. Also, all of total RNA separation from these cells or tissues, separation or isolation of mRNA, obtention and its cloning of cDNA can be conducted according to standard method. The screening method of gene of the invention among cDNA library includes methods used by a person skilled in the art such as method described in “Molecular Cloning 2nd Ed.”

In addition, mutant genes or homologous genes of the invention that are consisted of a nucleic acid sequence shown by any one of the (B) to (F) can be isolated by screening a homologue of the DNA from other organisms and the like under suitable conditions by using the nucleic acid sequence shown by SEQ ID NO: 1 or a DNA fragment having part of the nucleic acid sequence. Alternatively, it can be prepared using the method for producing a mutant DNA mentioned above.

Unsaturated fatty acids produced by fatty acid desaturase, which is expressed in fish body, varies depending on the type of the fatty acid desaturase used or fatty acid substrates used. However, docosahexaenoic acid (DHA) or eicosapentaenoic acid (EPA) can be suitably exemplified, and LNA (18:3n-3), OTA (18:4n-3), ETA (20:4n-3), DPA (22:5-3), TPA (24:5n-3), THA (24:6n-3), linoleic acid (18:2n-6), γ-linolenic acid (18:3n-6), eicosatrienoic acid (20:3n-6), arachidonic acid (20:4n-6) and docosapentaenoic acid (22:5n-6) are exemplified other than DHA (22:6n-3) and EPA (20:5n-3), but are not limiting.

As fish with increased unsaturated fatty acid content of the invention, they may originally have fatty acid desaturase gene or may not have it at all to the extent that fatty acid desaturase introduced is expressed and thereby increasing unsaturated fatty acid content in their body. However, fish subjected for aquaculture, fish in which unsaturated fatty acid content is increased selectively in edible portions such as muscle tissues, and fish in which fatty acid desaturase gene is stably expressed to their progeny are preferable. Teleost is preferred as type of fish. Especially, teleost belonging to Cyprinidae, Cichlidae, Salmonidae, Claridae, Siluridae, Ictaluridae are preferable. Specifically, cultured fish such as, but no limited to, buri (Seriola quinqueradiata), madai (Pagrus major), hirame (Paralichthys olivaceus), torafugu (Takifugu rubripes), hiramasa (Seriola aureovittata), kurumaebi (Panaeus japonicus), nijimasu (Oncorhyncus nykiss), kanpachi (Seriola dumerili), shimaaji (Pseudocaranx dentex), mahata (Epinephelus septemfasciatus), kuromaguro (Thunnus thynnus), and carp (for example Cyprinus carpio), zebrafish (Danino rerio), African catfish (Clarias gariepinus), tilapia (Oreochronis niloticus), Atlantic salmon (Salmo Salar) and medaka (Oryzias latipes) can be suitably specified.

For construction of an expression vector for introducing the fatty acid desaturase gene into fish, it is preferable to prepare an expression vector in which fatty acid desaturase gene is connected downstream of a promoter which efficiently expresses the fatty acid desaturase gene in fish cells. These promoters may include, but are not limited to, β-actin promoter, adipocyteP2 (aP2) promoter, Mylz2 (Danio rerio myosin light polypeptide 2 skeletal muscle mylz2) promoter, UCP promoter, SV40 promoter, cytomegavirus promoter, EF1αpromoter, Metallothionein promoter, heat shock promoter. However, medaka β-actin promoter and mylz2 promoter are especially preferable in regard to expression efficiency. In addition, it is preferable that a polyadenylation sequence such as a bovine growth hormone polyadenylation sequence is connected downstream of the fatty acid desaturase gene. In addition, an intron sequence and enhancer sequence that function to enhance gene expression, or a terminator sequence that directs termination of transcription may be used as appropriate. Introduction of the constructed expression vector into fish can be conducted by microinjection to oocytes or germ cells, virus vector infection, particle gun bombardment, or electroporation.

Transgenic fish of the invention include, but are not limited to, fish germ cells and fish embryo cells, other than adult fish in which the fatty acid desaturase gene is introduced and their progeny.

The invention will now be further described by way of the non-limiting examples which further illustrate the invention, and are not intended, nor should they be interpreted to, limit the scope of the invention.

### EXAMPLES

### Example 1

### Zebrafish Maintenance and Diets

The zebrafish used was AB strain (Walker et al., 1999). Fish were spawned and cultured as outlined by Westerfield (1995), with some modifications. Broodstock were raised in a 40-L aquaria under a photoperiod of 14 hours light and 10 hours dark at 28±1° C. Fish were fed on a commercial diet “otohime” (Nisshin Co.) and Artemia (Salt Creek Inc.) nauplii once daily, respectively. There were 4 aquariums for the broodstock, each containing 12 females and 8 males. These aquariums were divided into two groups, one group for spawning at 10:30 am and the other at 14:00 pm. This strategy allowed for microinjection to be carried out twice daily.

### Example 2

### Cloning of Δ6 Desaturase cDNA

The Δ6-desaturase-like cDNA was isolated from the liver of yamame salmon by PCR with two degenerated primers designed according to the GenBank database (AB070444). The primers used were (5′-TACTCCATGGTTCAGCAAATGAATTGAACA-3′; SEQ ID NO: 3) and (5′-TCGTCCATGGCCATTCACTGCTGACAAGGA-3′; SEQ ID NO: 4) for forward and reverse, respectively, both containing NcoI site (underlined) to facilitate cloning into the expression vector. PCR amplifications were performed under the following conditions: 94° C. for 3 min, followed by 30 cycles of 30 sec at 94° C., 30 sec at 62° C., and 1.5 min at 72° C. The amplified product of 1680 bp was then subcloned into pGEM-T Easy vector (Promega).

### Example 3

### Construction of Transgene Expression

The transgene expression was provided as an 8.5 kb plasmid pActD6 (FIG. 1). Briefly, the bovine growth hormone polyadenylation (BGH poly (A)) sequence was removed from the pRc/RSV (Invitrogen) by digestion with XhoI and ligated into pBluescript SK (+/−) (Clontech). The 3.7 kb medaka β-actin promoter (mβ-Actin) was modified by PCR from pOBA-109 (Takagi et al., 1994) to provide an EcoRI site for ligation into pBluescript SK (+/−) containing BGH poly (A). The 1477 kb Δ6-desaturase-like gene (D6D) of yamame salmon (O. masau) obtained from Example 2 was amplified by PCR with two oligonucleotide primers designed according to the GenBank database (AB070444). Using the forward primer “Sall-desF3” (5′-TTGTCGACGGTCTGAGTGGAGCAGAGAGAA-3′; SEQ ID NO: 5) containing a Sall recognition site (underlined), and the reverse primer “des-OmaR” (5′-ATCCAGGAAATGTCCTCTCTGTTCGCA-3′; SEQ ID NO: 6), PCR amplifications were performed under the following conditions: 94° C. for 3 min, followed by 30 cycles of 30 sec at 94° C., 30 sec at 62° C., and 1.5 min at 72° C. The PCR amplified products were cloned into the pGEM-T Easy vector (Promega), digested with Sall and then the Δ6-desaturase-like gene fragment was inserted between the medaka β-Actin promoter (mβ-Actin) and the BGH poly (A) sequence to produce the pActD6 construct.

### Example 4

### Preparation of DNA for Microinjection

Circular DNA obtained from Example 3 was diluted in a solution of 0.1 M KCl/0.125% tetramethyl-rhodamine dextran at a concentration of 30 μg/ml. The DNA solution was then filtered by an Ultrafree-MC Centrifuge Filter Devices 0.22 μm (MILLIPORE, Bedford) and spun at 5,000 rpm in a microcentrifuge at 4° C. for 5 min to remove any contaminating particulates. Three to four μL of DNA was transferred onto a clean glass cover and overlaid with a few drops of mineral oil to prevent evaporation.

### Example 5

### Preparation of Microinjection Plates

To make a microinjection plate, a mold was placed onto a 90-mm Petri dish and a warm 3% agarose (prepared in distillated water) of about 30 ml was poured into the plate. After the agarose has solidified at room temperature, the mold was removed to make twelve grooves. These grooves are to support embryos when penetrated by microinjection needle. The used plate was then covered with plastic wrap and stored at 4° C. until use.

### Example 6

### Preparation of the Microinjection Needle

Microinjection needles were made with Micropipette Puller (Narishige; FIG. 2A) using a fine-glass microinjection capillary. The tip of the microinjection needle used was about 5-8 μm. The needle is attached to the holder and filled with mineral oil from the syringe by turning micromanipulator to the right-hand, through the Teflon tubing, and into the needle.

### Example 7

### Microinjection Set Up

The microinjection system used consists of a stereomicroscope, a micromanipulator prepared in Example 6 for controlling the microinjection needle during the microinjection process, a magnetic stand associated to the micromanipulator (Narishige) for INA loading, a plastic two-way stopper, Teflon tubing (Narishige), and a microinjection plate. The micromanipulator with a needle holder for DNA loading was attached to the magnetic stand which was firmly fastened to a metal surface. The syringe was filled with 3 ml of mineral oil and was directly connected to the two-way stopper. This stopper was connected to about 40 cm of 1-mm Teflon tubing that was in turn connected to the microinjection needle holder.

### Example 8

### Collection of Eggs and Microinjection

On the day of microinjection, 3 males and 2 females of zebrafish described in example 1 were transferred from their aquarium to the 3-L breeding tank at least 15 minutes before the lamp came on. Eggs were collected in a Petri dish approximately 5 minutes after being laid. The 1-cell-stage embryos were then gently transferred onto the groove of the microinjection plate prepared in Example 5 using a pipette. Blastodisc should be facing the microinjection needle. The DNA solution prepared in Example 4 was microinjected into the cytoplasm of 1-cell-stage embryos on the microinjection plate using the microinjection system prepared in Example 7. Embryos that had been injected with DNA were kept at 28±1° C. Unfertilized and deformed eggs were removed 5-6 hours after fertilization. Next day, dead and deformed embryos were removed and healthy embryos were transferred into new Petri dishes.

### Example 9

### RNA Isolation and Semi-Quantitative RT-PCR

Samples of twenty embryos are taken at 8 hours post fertilization to isolate mRNA in order to quantify the transcript levels. The total RNA was isolated by ISOGEN (Nippon Gene) according to the manufacturer's instructions. Any traces of DNA were degraded with RQ1 RNase-free Dnase (Promega) 2 U/μg for 30 min at 37° C. After phenol-chloroform extraction and ethanol precipitation, pellets were dissolved in diethylpyrocarbonate (DEPC)-treated water. Single-stranded cDNA was synthesized from 2-3 μg of total RNA with Ready-to-Go You-Prime First-Strand Beads (Amersham Pharmacia Biotech) according to the manufacturer's instructions. In this reaction dT3RACE-VECT primer (5′-GTAATACGAATAACTATAGGGCACGCGTGGTCGACGGCCCGGGCTGG(T)-3′; SEQ ID NO: 7) was used.

Semi-quantitative RT-PCR was conducted to quantify the transcript levels of foreign desaturase gene. PCR analysis was performed in 20 μl of 10×Ex Taq Buffer, 200 μM of dNTPs, 0.125 U of Ex Taq polymerase (Takara), 2 μl of cDNA as template and 1 pmol of each under the following conditions: 94° C. for 3 min, followed by 24 cycles of 30 sec at 94° C., 30 sec at 62° C., and 1.5 min at 72° C. The sequences of the forward primer for desaturase and the reverse primer were as follows: “Omar” (5′-AGGACTGGCTCACCATGCAGTTGAGT-3′; SEQ ID NO: 8) and the above-mentioned “des-Omar” (SEQ ID NO: 4). The β-actin gene expression was also analyzed as an internal control of equal loading RNA. The forward primer for β-actin gene was (5′-ACTACCTCATGAAGATCCTG-3′; SEQ ID NO: 9) and the reverse primer was (5′-TTGCTGATCCACATCTGCTG-3′; SEQ ID NO: 10). Two μl from the reaction was electrophoretically separated using 0.7% agarose gel, stained with ethidium bromide, and photographed under ultraviolet light. Fluorescence intensity of each band was quantified by Densitograph (Atto Co. ltd.). The construct showing the strongest expression was then used to produce stable lines.

The desaturase gene expression of F1 and F2 progeny were also analyzed by the method used for F0. Total RNA was extracted from the fin, liver and muscle of one DNA-positive fish to identify foreign gene expression in F1. The total RNA in F2 generation was extracted from at least twenty 2-day-old larvae, and from muscle and liver of at least 10 adult fish.

### Example 10

### DNA Isolation and PCR Amplification

Identification of transgenic individuals was conducted by PCR with DNA template that had been extracted from caudal fin tissue. The DNA was also extracted from 2-day-old pooled-larvae obtained by crossing the DNA-positive F0 with non-transgenic fish in order to identify the germline transmitters. Isolation of DNA genome was performed using a DNA Isolation Kit (Puregene) according to the manufacturer's instructions. PCR reactions and primers used were the same as the method used for RT-PCR.

### Example 11

### Production of Stable Lines

The DNA-positive F0 were raised to adult fish. The matured F0 was crossed with a non-transgenic zebrafish, and at least twenty 2-day-old larvae from the cross were pooled for screening the germline transmitters. The germline-positive F0 were then used to produce F1 and F2 generations. Production and screening procedures for F1 and F2 were the same as the method used for F0.

### Example 12

### Fatty Acid Analysis

The total lipids were extracted from non-transgenic and transgenic fish containing the masou salmon Δ6-desaturase gene. The non-transgenic and transgenic fish were maintained in the same aquarium, and total fatty acid was extracted from total fish. Fatty acids were separated using a gas chromatography (Shimadzu 14B; Shimadzu) equipped with a hydrogen flame ionization detector and a capillary column of 30 m×0.32 mm×0.25 μm (Supelco), and the fatty acid methyl esters (FAMEs) were prepared from fatty acids by the method described previously (references). With the measurement with gas chromatography, fatty acids methyl ester peaks were identified by comparison of their retention times with an appropriate FAME standard (Supelco).

### Example 13

### Production of Stable Line and Results

Transient foreign gene expression was analyzed with cDNA synthesized from RNA extracted from the injected embryos at 8 hours post fertilization. The results that indicate transient expression are shown in FIG. 2. Also the results that identified foreign gene expression in F1 extracted from fin, liver and muscle of DNA-positive fish are shown in FIG. 3. The results of the experiment conducted with the same conditions except using Mylz2 promoter instead of medaka β-actin promoter are also shown in FIG. 3. FIG. 3 indicates that foreign gene expression was observed in fin and muscle in both cases using medaka β-actin promoter or Mylz2 promoter.

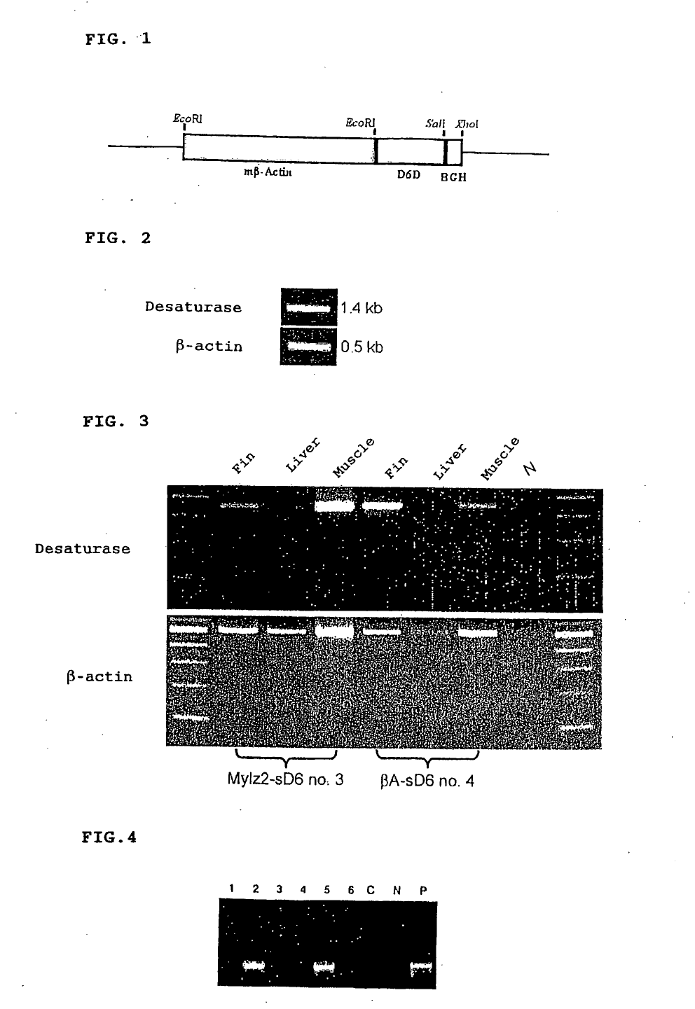

The transgenic individuals were screened by PCR amplification of genomic DNA extracted from fin using primers specific for the masou salmon desaturase gene. The DNA-positive fish was crossed with non-transgenic to screen the germline transmitter's fish and the results are shown in Table 1 and FIG. 4. As shown in Table 1, among 400 embryos injected with DNA, 109 were raised and 4 out of the 109 adult fish carried pActD6 gene construct in their germ cells. These fish were then used to produce F1 and F2 generations. The transmission rate of foreign gene into F1 generation was varied between 4.2% and 44.1% as shown in Table 2. These results confirm that transgenic F0 fish were mosaic. The F1 transgenic line which has higher mRNA transcription level analyzed by RT-PCR was used to produce F2 generation. Representative foreign gene expression is shown in FIG. 5. The transmission of transgene into F2 generation followed a Mendelian segregation pattern. These results confirmed that all diploid cells of each F1 transgenic line contained transgene.

TABLE 1

| Embryos | | | DNA-positive | Germline |
|---|---|---|---|---|
| injected | Hatch | Adult stage | fish | transmitter |
| 400 | 178 | 109 | 16 | 4 (3.7%) |

TABLE 2

Inheritance of foreign gene

| No. line (F0) | F1 generation | | F2 generation |
|---|---|---|---|
| 10 | 39.1% | (9/23) | 46.4% (45/97) |
| | | | 44.4% (55/124) |
| 12 | 44.1% | (15/34) | n.a. |
| 13 | 20.0% | (20/100) | n.a. |
| 15 | 4.2% | (5/120) | 56.7% (17/30) |
| | | | 50.0% (19/30) |
| | | | 60.0% (18/30) |

### Example 14

### Fatty Acid Composition in Transgenic Fish

Fatty acid methyl esters (FAMEs) were prepared both from whole body of non-transgenic and transgenic fish and analyzed by gas chromatography (GC). The n-3 fatty acids compositions of transgenic fish are shown in Table 3. The EPA and DHA production in transgenic fish expressing the masou salmon desaturase gene was 1.4±0.1 fold (1.86±0.03 mg/g vs 1.32±0.05 mg/g) and 2.1±0.2 fold (4.62±0.22 mg/g vs 2.22±0.08 mg/g) higher (p<0.05) than that of non-transgenic counterparts. However, there was highly varied amount of EPA and DHA production between transgenic lines. The amount of EPA and DHA production in edible part was about 75% and 78% to whole body of the fish, respectively.

TABLE 3

| | | | | | | Total of |
|---|---|---|---|---|---|---|
| | n-3 fatty acids composition | | | | | crude lipid |
| Fish | 18:3n-3 | 18:4n-3 | 20:4n-3 | 20:5n-3 | 22:6n-3 | (% wet weight) |
| Non-transgenic | 5.30 ± 0.29 | 0.54 ± 0.02 | 0.30 ± 0.00 | 1.32 ± 0.05 | 2.22 ± 0.08 | 5.64 ± 0.30 |
| | (9.39 ± 0.03) | (0.96 ± 0.01) | (0.54 ± 0.03) | (2.34 ± 0.05) | (3.95 ± 0.14) | |
| pActD6 | 5.91 ± 0.25 | 0.63 ± 0.03 | 0.44 ± 0.04 | 1.70 ± 0.25 | 3.01 ± 0.09 | 6.17 ± 0.22 |
| 10-1 | (9.58 ± 0.09) | (1.02 ± 0.02) | (0.71 ± 0.05) | (2.74 ± 0.31) | (4.88 ± 0.16) | |
| pActD6 | 4.30 ± 0.15 | 0.43 ± 0.02 | 0.42 ± 0.01 | 1.86 ± 0.03 | 4.62 ± 0.22 | 5.53 ± 0.13 |
| 15-2 | (7.77 ± 0.17) | (0.78 ± 0.03) | (0.75 ± 0.01) | (3.37 ± 0.06) | (8.36 ± 0.42) | |

The results shown in Table 3 are means±SD of triplicate experiments. SD=0.0 implies an SD<0.005. Values within parenthesis are percentage of each fatty acid of total fatty acids. Non-transgenic and transgenic fish was maintained in the same aquarium. Total fatty acids were extracted from whole fish, with fatty acid methyl esters having been analyzed and quantified by GC. Numbers at the end of construction name indicate individual transgenic line.

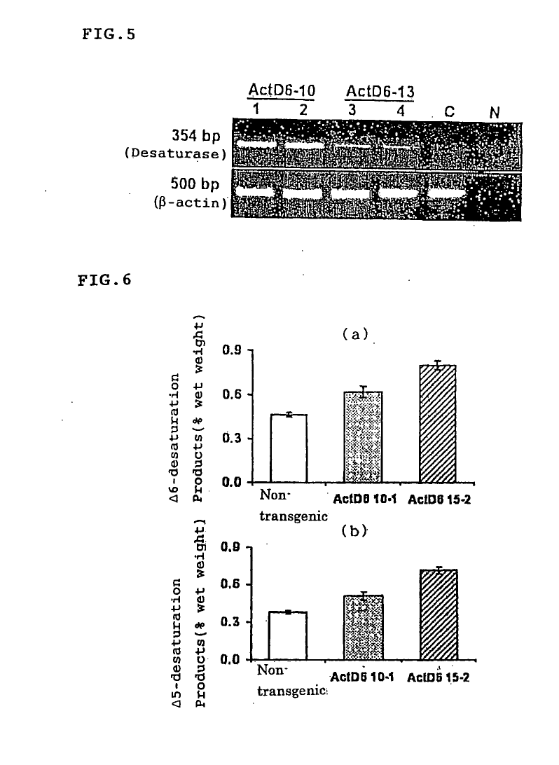

To confirm whether the introduced gene is Δ6- or Δ5-desaturase, the fatty acids except the substrates of desaturation was summed. As shown in FIG. 6, the Δ6- and Δ5-desaturation products of transgenic fish was higher (p<0.05) than that of non-transgenic fish. The higher amount of fatty acid products of Δ6-desaturation indicating the introduced gene is attributable to a foreign Δ6-desaturase.

The invention is further described by the following numbered paragraphs:

1. Transgenic fish with increased unsaturated fatty acid content, wherein a fatty acid desaturase gene is introduced into the transgenic fish and the fatty acid desaturase gene is expressed therein.

2. The transgenic fish according to paragraph 1, wherein the fatty acid desaturase gene is connected downstream of β-actin promoter derived from medaka.

3. The transgenic fish according to paragraph 1 or 2, wherein the fatty acid desaturase gene is connected upstream of a bovine growth hormone polyadenylation sequence.

4. The transgenic fish according to any one of paragraphs 1 to 3, wherein the fatty acid desaturase gene which consists of any one of the nucleic acid sequences of following (A) to (F);

(A) Nucleic acid sequence encoding a protein which consists of the amino acid sequence set forth by SEQ ID NO: 2;

(B) Nucleic acid sequence that encodes a protein consisting of an amino acid sequence with deletion, substitution or addition of one or more amino acids in the amino acid sequence set forth by SEQ ID NO: 2 and said protein having a fatty acid desaturase activity;

(C) Nucleic acid sequence that encodes a protein consisting of an amino acid sequence having not less than 60% of homology with the amino acid sequence shown by SEQ ID NO: 2, and said protein having a fatty acid desaturase activity;

(D) Nucleic acid sequence shown by SEQ ID NO: 1;

(E) Nucleic acid sequence that consists of a nucleic acid sequence with deletion, substitution or addition of one or more bases in the nucleic acid sequence shown by SEQ ID NO: 1 and that encodes a protein having a fatty acid desaturase activity;

(F) Nucleic acid sequence that hybridizes to the nucleic acid sequence shown by SEQ ID NO: 1 under stringent conditions and that encodes a protein having a fatty acid desaturase activity.

5. The transgenic fish according to any one of paragraphs 1 to 4, wherein the unsaturated fatty acid is eicosapentaenoic acid (EPA) or docosahexaenoic acid (DHA).

6. The transgenic fish according to any one of paragraphs 1 to 5, wherein the transgenic fish are cultured fish.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. Transgenic fish with increased unsaturated fatty acid content, wherein a fatty acid desaturase gene is introduced into the transgenic fish and the fatty acid desaturase gene is expressed therein.

2. The transgenic fish according to claim 1, wherein the fatty acid desaturase gene is connected downstream of β-actin promoter derived from medaka.

3. The transgenic fish according to claim 1, wherein the fatty acid desaturase gene is connected upstream of a bovine growth hormone polyadenylation sequence.

4. The transgenic fish according to claim 1, wherein the fatty acid desaturase gene consists of a nucleic acid sequence encoding a protein, wherein said protein comprises the amino acid sequence shown by SEQ ID NO: 2.

5. The transgenic fish according to claim 4, wherein the nucleic acid sequence encoding a protein comprises an amino acid sequence with deletion, substitution or addition of one or more amino acids in the amino acid sequence shown by SEQ ID NO: 2, and said protein has a fatty acid desaturase activity.

6. The transgenic fish according to claim 5, wherein the nucleic acid sequence encoding a protein comprises an amino acid sequence having not less than 60% of homology with the amino acid sequence shown by SEQ ID NO: 2, and said protein has a fatty acid desaturase activity.

7. The transgenic fish according to claim 1, wherein the fatty acid desaturase gene consists of a nucleic acid sequence shown by SEQ ID NO: 1.

8. The transgenic fish according to claim 7, wherein the nucleic acid sequence comprises a nucleic acid sequence with deletion, substitution or addition of one or more bases in the nucleic acid sequence shown by SEQ ID NO: 1, and that encodes a protein having a fatty acid desaturase activity.

9. The transgenic fish according to claim 7, wherein the nucleic acid sequence hybridizes to the nucleic acid sequence shown by SEQ ID NO: 1 under stringent conditions and encodes a protein having a fatty acid desaturase activity.

10. The transgenic fish according to claim 1, wherein the unsaturated fatty acid is eicosapentaenoic acid (EPA) or docosahexaenoic acid (DHA).

11. The transgenic fish according to claim 1, wherein the transgenic fish are cultured fish.


SEQUENCE LISTING (10 sequences)

SEQ ID NO: 1 - DNA, 1692 bp, Oncorhynchus masou
tgtcattcgatattttcagcaaatgaattgaacaacacattttgttgtacaacgctgtctggaaaacatctcccttccctctggcagaagaatcgcagtgtgtttcagttggacagactggacagagtgcagccgacacagcgacggtgtgagtggagcagagagaaccgaggatggggggcggaggtcagcagacggagtcaagcgagccggccaagggtgacggggttgggcccgatggagggcgaggtggcagtgcagtctacacctgggaagaggtccagaagcactgccacagaagcgaccagtggttggtcatcgacaggaaggtctataatattacccagtgggcgaagagacacccagggggcatcagggtcatcagtcactttgctggagaagatgccacggaagcatttgtcgcattccatctcgaaccgaattttgtcaggaagtttctgaagccgttgctgattggagagctggcaccgacagagcccagccaggaccaggggaaaaatgcagtactggtgcaggacttccaggccctgcgtgaccgtgtggagagtgagggtctcctccgtgcccgccccctcttcttcagcctctacctgggccacatcctgctactagaggccctggctttgggcctgctctgggtctgggggaccagctggagcctcacactgctctgttccctcatgctggccacgtctcaggcccaggctggctggctgcagcatgactacggccacctgtcagtctgcaagaaatctggctggaaccacaaactgcacaagtttgtcattggacacctaaagggtgcctctgctaactggtggaaccatcgtcacttccagcaccacgctaagcccaacgtgtttagtaaagatcctgatatcaactcactgcatgtcttcgtcctgggagacaaacagcctgtagagtatggtataaagaagttgaagtacatgccctaccatcaccaacaccagtacttcttcctcgttggacctccactcatcgttccagtgtttttcaacatccagatattccggaccatgttttcacaacgagactgggtggatctggcgtgggcgatgactttctaccttcgcttcttctgctgttactatcccttctttggtttctttggctcagtagcattgatcagcttcgtcaggtttttggaaagccactggtttgtatgggtgacccagatgagtcaccttccgatggagatggatcacgagagacaccaggactggctcaccatgcagttgagtgctacttgcaacattgaacagtcaaccttcaacgactggttcagtggacacctcaactttcagattgaacaccatctgtttcctaccatgccccgtcataactaccacctggtagctcctctggttcgtgctttgtgtgagaaacatggagttccctaccaggtcaagactttgcagaaaggcatgactgatgttgtcaggtcactgaagaagtcaggggatctgtggctggatgcgtatctccataaataaatcccttcctgactctggacgggatttaatccatcgcagattaacgacctgcgaacagagaggacatttcctggatgtttttgatgataatgatcgctgcaaacattattgtgatataattgttttaatccttgtcagcagtgaatggggatcc

SEQ ID NO: 2 - protein, 454 aa, Oncorhynchus masou
MGGGGQQTESSEPAKGDGVGPDGGRGGSAVYTWEEVQKHCHRSDQWLVIDRKVYNITQWAKRHPGGIRVISHFAGEDATEAFVAFHLEPNFVRKFLKPLLIGELAPTEPSQDQGKNAVLVQDFQALRDRVESEGLLRARPLFFSLYLGHILLLEALALGLLWVWGTSWSLTLLCSLMLATSQAQAGWLQHDYGHLSVCKKSGWNHKLHKFVIGHLKGASANWWNHRHFQHHAKPNVFSKDPDINSLHVFVLGDKQPVEYGIKKLKYMPYHHQHQYFFLVGPPLIVPVFFNIQIFRTMFSQRDWVDLAWAMTFYLRFFCCYYPFFGFFGSVALISFVRFLESHWFVWVTQMSHLPMEMDHERHQDWLTMQLSATCNIEQSTFNDWFSGHLNFQIEHHLFPTMPRHNYHLVAPLVRALCEKHGVPYQVKTLQKGMTDVVRSLKKSGDLWLDAYLHK

SEQ ID NO: 3 - DNA, 30 bp, Artificial Sequence
  Description of Artificial Sequence Synthetic primer
tactccatggttcagcaaatgaattgaaca

SEQ ID NO: 4 - DNA, 30 bp, Artificial Sequence
  Description of Artificial Sequence Synthetic primer
tcgtccatggccattcactgctgacaagga

SEQ ID NO: 5 - DNA, 30 bp, Artificial Sequence
  Description of Artificial Sequence Synthetic primer
ttgtcgacggtctgagtggagcagagagaa

SEQ ID NO: 6 - DNA, 27 bp, Artificial Sequence
  Description of Artificial Sequence Synthetic primer
atccaggaaatgtcctctctgttcgca

SEQ ID NO: 7 - DNA, 48 bp, Artificial Sequence
  Description of Artificial Sequence Synthetic primer
gtaatacgaataactatagggcacgcgtggtcgacggcccgggctggt

SEQ ID NO: 8 - DNA, 26 bp, Artificial Sequence
  Description of Artificial Sequence Synthetic primer
aggactggctcaccatgcagttgagt

SEQ ID NO: 9 - DNA, 20 bp, Artificial Sequence
  Description of Artificial Sequence Synthetic primer
actacctcatgaagatcctg

SEQ ID NO: 10 - DNA, 20 bp, Artificial Sequence
  Description of Artificial Sequence Synthetic primer
ttgctgatccacatctgctg